(19) United States
(12) Patent Application Publication
(10) Pub. No.: US 2026/0345353 A1
(43) Pub. Date: Nov. 5, 2026

(54) TRANSMISSION

(71) Applicant: OTTO BOCK HEALTHCARE PRODUCTS GMBH, Wien (AT)

(72) Inventors: Wolfgang SAUBERER, Wien (AT); Christoph ZAHALKA, Wien (AT)

(21) Appl. No.: 19/488,350

(22) PCT Filed: May 29, 2024
(86) PCT No.: PCT/EP2024/064729

§ 371 Date: Nov. 26, 2025

(30) Foreign Application Priority Data
May 30, 2023 (DE) 10 2023 114 122.8

(51) Int. Cl. A61F 2/68

(52) U.S. Cl. CPC A61F 2/68; A61F 2002/6836

(57) ABSTRACT

The invention relates to a transmission for orthopedic devices, with a planetary gear set (10), which has a central sun gear (11), with a plurality of planet gears (12) which are rotatingly mounted on a planet carrier (13) and are force-transmittingly coupled to the sun gear (11), and with a ring gear (14), which has an inner circumference, in which the planet gears (12) circulate, the sun gear (11) is coupled to a drive shaft (2), and the planet carrier (13) is coupled to an output element (20), wherein the output element (20) is coupled to two contradirectionally oriented, shiftable freewheels (30, 40), wherein, in a first direction of rotation of the drive shaft (2), a first freewheel (30) can be brought directly into force-transmitting engagement with the drive shaft (2), and, in a second direction of rotation of the drive shaft (2), counter to the first direction of rotation, a second freewheel (40) can be brought into force-transmitting engagement with the planet carrier (13), in order to drive the output element (20).

The invention relates to a transmission for orthopedic devices, with a planetary gear set, which has a central sun gear, with a plurality of planet gears which are rotatingly mounted on a planet carrier and are force-transmittingly coupled to the sun gear, and with a ring gear, which has an inner circumference, in which the planet gears circulate, the sun gear is coupled to a drive shaft, and the planet carrier is coupled to an output element.

Orthopedic devices, in particular orthoses, exoskeletons and prostheses, often have components that are mounted pivotably or displaceably on one another. Various measures are taken to influence the relative movement between the components, for example an upper part and a lower part of an orthosis spanning a joint or of a prosthesis with a joint. Mechanical stops can be provided to limit the maximum pivoting angle or displacement travel. In order to influence the pivoting movement or displacement movement, force accumulators or dampers can be arranged as passive influencing devices between the two components.

Active orthopedic devices have a drive which initiates and assists a relative movement between the components or offers a resistance to the relative movement. Electric motors are often provided for this purpose, which are activated, deactivated or modulated via a control device. The electric motor is coupled to an accumulator or battery in order to permit appropriate active influencing or initiation of a relative movement.

Both for the energy store and for the drive in the form of motors, the space that is available in orthopedic devices is limited. Motors that are able to generate high torques are often too heavy, especially in orthopedic devices of the upper extremities. Therefore, small, high-speed motors are generally used, which are coupled via a transmission to the respective component that is to be driven or influenced.

A shift clutch for prostheses, especially for prosthetic gripping prostheses, is known from U.S. Pat. No. 8,157,446 B1. The shift clutch has a chassis on which at least one finger prosthesis is hinged. The shift clutch also has a drive element, an output element and a shifting device, which switches between two gear stages depending on the torque applied to the output element. The shifting device operates independently of the direction of rotation of the output element and switches between the gear stages with different ratios. This is effected via clamping bodies which are in contact with the shifting device and which couple the output element to the drive element in a torque-controlled manner.

The requirements for a drive train of an active orthopedic device are numerous and depend in particular on the design of the orthopedic device and on the manner of use. In orthopedic devices of the lower extremities, a distinction can be made in principle between use in the swing phase and use in the stance phase. Lower torques and a high adjustment speed are generally required in the swing phase, whereas high torques and comparatively low adjustment speeds are required in the stance phase. In the case of an active knee joint, for example, only small adjustment torques of below 10 Nm are required in the swing phase initiation, whereas, in a stance phase extension, high torques of greater than 30 Nm are required at low knee angle velocities of below 100°/s in the extension direction. When designing and optimizing the drive train, it is therefore necessary to meet a wide range of requirements, although it is difficult with conventional transmissions to optimize the drive train in such a way that the different operating points are optimally achieved.

The problem addressed by the present invention is to provide a transmission in order to be able to operate the drive of an orthopedic device at an optimal operating point, so as to meet different requirements with regard to the adjustment speed and the adjustment moments.

This problem is solved by a transmission having the features of the main claim. Advantageous embodiments and developments of the invention are explained in more detail in the dependent claims, the description and the figures.

The transmission for an orthopedic device, with a planetary gear set, which has a central sun gear, with a plurality of planet gears which are rotatingly mounted on a planet carrier and are force-transmittingly coupled to the sun gear, and with a ring gear, which has an inner circumference, in which the planet gears circulate, wherein the sun gear is coupled to a drive shaft, and the planet carrier is coupled to an output element, is characterized in that the output element is coupled to two contradirectionally oriented, shiftable freewheels, wherein, in a first direction of rotation, a first freewheel can be brought directly into force-transmitting engagement with the drive shaft, and, in a second direction of rotation, counter to the first direction of rotation, a second freewheel can be brought into force-transmitting engagement with the planet carrier, in order to drive the output element. With such a transmission, it is possible to operate the drive at a good operating point and thus with a high degree of efficiency in different load situations depending on the direction of rotation, such that the energy consumption can be reduced and the drive as a whole can be dimensioned smaller. This saves both installation space and weight, as a result of which the overall system of the orthopedic device is improved. The transmission makes it possible to achieve high speeds in one direction of adjustment, while in the opposite direction of adjustment a high torque can be provided at lower speeds of adjustment. By virtue of the shiftable freewheels, the output element is either brought directly into force-transmitting engagement with the drive shaft or in the other direction of rotation via the planetary gear mechanism, as a result of which different ratios are set depending on the direction of rotation. In the first direction of rotation, there is a direct coupling between the drive shaft and the output element so that, for example, the speed of the motor is transferred directly to the output element, while in the second direction of rotation there is a ratio of 1 to 6 for example.

In one embodiment, the freewheels have clamping bodies which, in an engagement position, come into force-transmitting engagement with the output element and the drive shaft or the planet carrier, and the clamping bodies are assigned shifting pins which move the clamping bodies from the engagement position to a release position, in which the clamping bodies are disengaged, and back. Both freewheels have clamping bodies that are arranged between the output element and the drive shaft or a component driven by the drive shaft. The clamping bodies slide or roll along the surface of the drive shaft or of a component driven by the drive shaft, in particular along the planet carrier, and clamp with the output element depending on the direction of rotation. The driven component carries the clamping body along in the respective direction of rotation. In the engagement position, the drive shaft or planet carrier is clamped with the output element, so that the output element can be driven by the drive shaft. In the opposite direction of rotation, on account of the given geometric conditions between the clamping bodies, the output element and the drive shaft or the planet carrier for the respective freewheel, no force transmission from the drive shaft to the output element is possible, or it is so slight that no rotation of the output element takes place. In order to bring the clamping bodies into a position in which they are disengaged from the output element and from the drive shaft or from the component driven by the drive shaft, the clamping bodies are assigned shifting pins, via which the clamping bodies can be moved from the engagement position to a release position. For example, the clamping bodies are moved away from the surface of the drive shaft or the driven component, in particular the planet carrier, or a clearance is created between the clamping bodies and the drive shaft or the planet carrier, so that no force transmission takes place. The shifting pins, which can be actuated, make it possible in particular to engage or disengage the clamping bodies in a controlled manner, which also increases the precision of the transmission. If the clamping bodies have already been brought into engagement with the output element and, for example, with the drive shaft by the shifting pins, the freewheels are activated in the corresponding direction of rotation with less play or idle travel. Conversely, if the clamping bodies have already been adjusted from the engagement position to the release position, a reversal of the direction of rotation is facilitated, since the clamping bodies no longer have to be brought to the release position by the drive.

In one embodiment, the shifting pins, in particular all of the shifting pins, are arranged on a rotatably mounted carrier. By a rotation of the carrier about an axis of rotation, it is possible to activate or deactivate all of the clamping bodies simultaneously or to engage or disengage them with/from the drive element and the drive shaft or the planet carrier.

In one embodiment, the freewheels are arranged coaxially to each other and arranged axially one behind the other, so that a compact design can be realized. The diameters of the freewheels can be different; in principle, it is also possible to have the same diameters for the freewheels so that, if necessary, identical freewheels can be used axially staggered one behind the other. On at least one shifting pin there are arranged both a clamping body for the first freewheel and a clamping body for the second freewheel, wherein the two clamping bodies for the first freewheel and the second freewheel are in particular formed separately. The shifting pin or all of the shifting pins, in particular the shifting pins on the rotatably mounted carrier, protrude axially both into the first freewheel and into the second freewheel and allow the clamping bodies to be moved from an engagement position to a release position and back again by a rotational movement in one direction or in the other. To effect the adjustment, an actuator is preferably assigned to the carrier; in particular, the carrier is coupled to the actuator. The actuator can be designed as an active drive, in particular as an electric motor or another electric or electronically activatable, deactivatable and/or modulatable drive, which can also be designed, for example, as a lockable or activatable force accumulator, for example a spring.

In one embodiment, the clamping bodies and the carrier are designed in such a way that all of the clamping bodies are held in the release position. The carrier with the shifting pins is thus able to adopt a position in which the clamping bodies are in an engagement position neither in the first freewheel nor in the second freewheel. In this position, all of the clamping bodies are in the release position, so that a complete decoupling of the drive shaft from the output element can be achieved.

In one embodiment, the first freewheel is formed in a sleeve-shaped section of the output element. The clamping bodies of the first freewheel are arranged on, and slide or roll on, the outer side of an output section arranged or formed on the drive shaft. Outside the clamping bodies there is then arranged a sleeve-shaped part of the output element in which, in one direction of rotation of the drive shaft, the clamping bodies are clamped and, in the other direction, can roll or slide without transmission of force to the output element. The output section can be formed in one piece on the drive shaft; alternatively, the output section is formed as a separate assembly, which is driven by the drive shaft and displaced in the corresponding direction of rotation.

In one embodiment, the output element is rotatably mounted on an inner circumference of the planet carrier; in particular, the output element is sleeve-shaped and protrudes into the inner circumference of the planet carrier. The clamping bodies of the second freewheel are arranged between the inner side of the output element and an outer side of a sleeve section of the planet carrier. The output element is, for example, mounted in roller bearings or ball bearings within the planet carrier, wherein the bearings are arranged on the outer side of the output element and on the inner side of the planet carrier in an inner circumference, for example in an annular space. By way of the clamping bodies, the planet carrier, in an engagement position on the inner side of the output element, is then brought into clamping and force-transmitting contact with the drive shaft and the output element.

The planetary gear set can be designed as a friction gear or gearwheel mechanism. In an embodiment as a gearwheel mechanism, external teeth are provided for the sun gear and the planet carrier; internal teeth are then formed on or in the ring gear.

In one embodiment, receiving pockets are arranged or formed in the output element and increase radially in size in a circumferential direction. The clamping elements are located in the receiving pockets, wherein the increase in size of the receiving pockets in the radial direction is dimensioned in such a way that, at least in the end positions as seen in the circumferential direction, a release position without relevant force transmission or an engagement position in which the output element is or can be coupled to the drive shaft with a clamping action, optionally directly or via the planet carrier. The increase in size of the receiving pockets for the clamping elements of the first freewheel runs counter to the increase in size of the second freewheel. The freewheels are thus oriented in opposite directions and bring about a direction-dependent and different ratio. In order to be able to decouple the drive shaft completely from the output element, the receiving pockets for the clamping elements are dimensioned and positioned in such a way that, in particular by way of the carrier, the clamping elements can be disengaged both from the first freewheel and the second freewheel, so that forces can be transmitted neither in one direction nor in the other direction.

To be able to bring about a radial displacement of the clamping bodies relative to the output element or to the drive shaft, to a component connected to the latter, or to the planet carrier, the clamping bodies have oblong holes, which are used to receive the shifting pins. In one embodiment, each clamping body has a single oblong hole so that, in particular when the carrier is rotated in the direction of an engagement position, the clamping bodies lie with their outer circumference on the receiving pockets and, upon rotation in the circumferential direction relative to the corresponding freewheel, the clamping bodies move radially inward in the direction of the planet carrier or the drive shaft, until there is a contact between the clamping bodies and the corresponding surface of the drive shaft or planet carrier sufficient to carry the clamping bodies further and clamp them.

An exemplary embodiment of the invention is explained in more detail below on the basis of the figures. The same reference signs in different figures refer to the same components. To ensure clarity, not all of the reference signs are indicated in all of the drawings. In the figures:

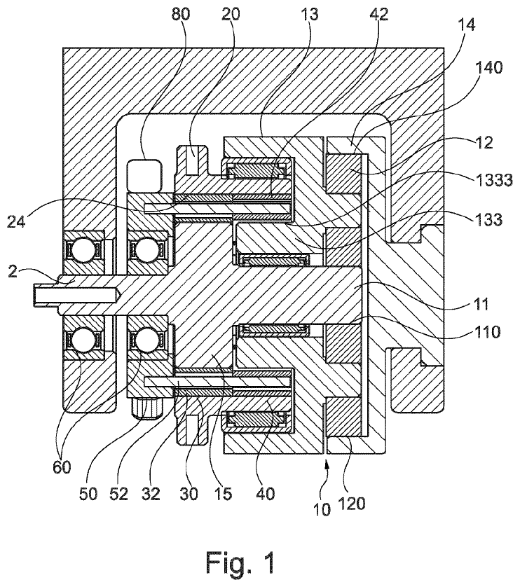

FIG. 1 shows a sectional view through a transmission;

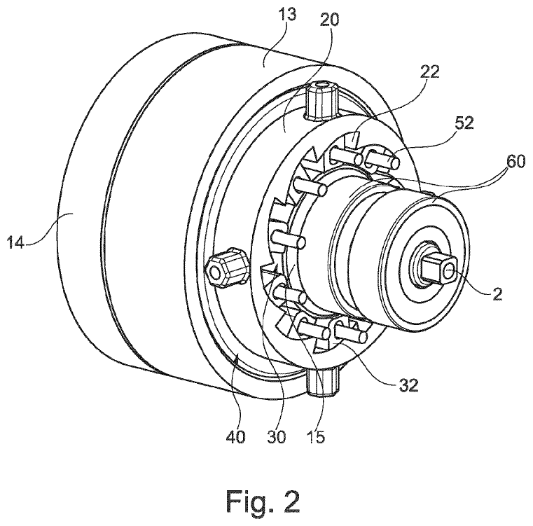

FIG. 2 shows a perspective view of the transmission with some of the components removed;

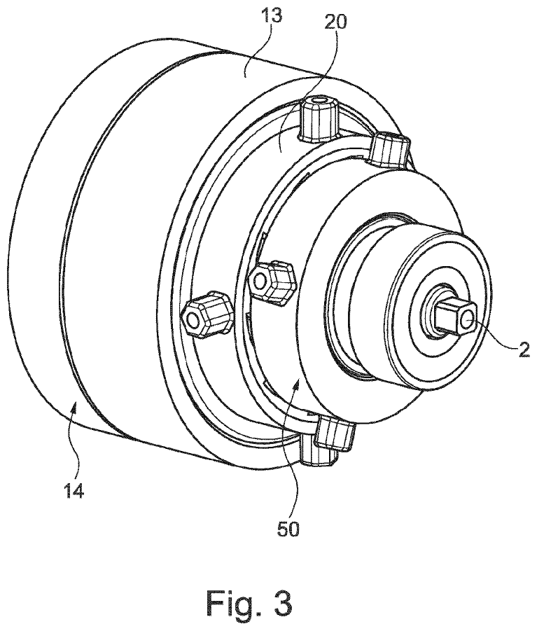

FIG. 3 shows FIG. 2 with a carrier;

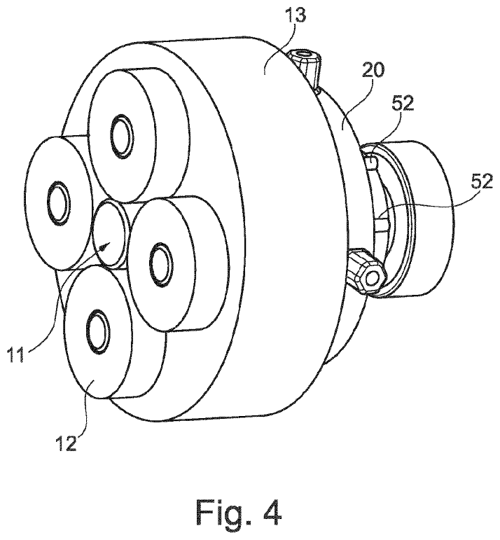

FIG. 4 shows a view from a different perspective, without a ring gear;

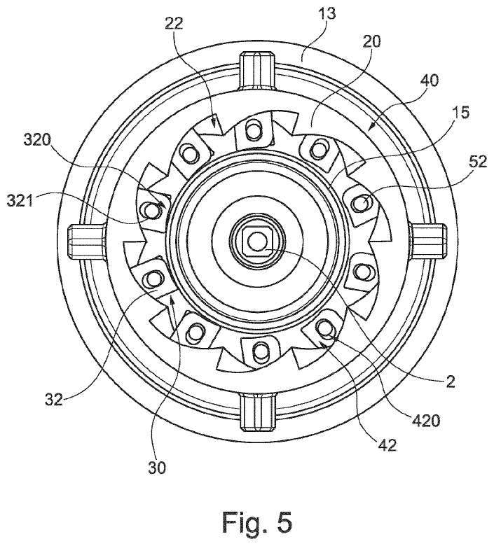

FIG. 5 shows a front view of an embodiment; and

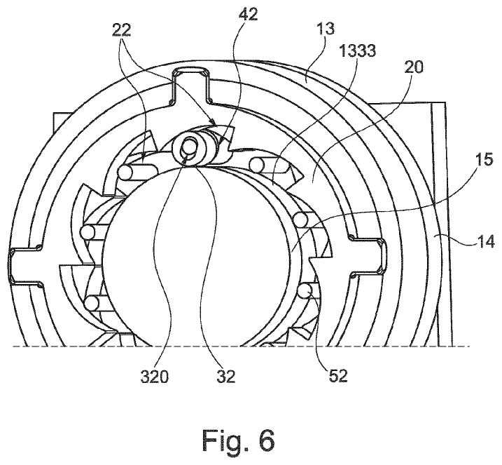

FIG. 6 shows a variant of FIG. 5.

In FIG. 1, a transmission for orthopedic devices, with a planetary gear set 10, is shown in a sectional view. The planetary gear set 10 has a central sun gear 11, which has an external toothing 110 and, in the exemplary embodiment shown, is designed as part of a drive shaft 2. The drive shaft 2 is driven by a motor, either directly or via an intermediate gear system. As an alternative to the one-piece design of drive shaft 2 and sun gear 11, the sun gear 11 can also be torque-transmittingly fastened to the drive shaft 2. The sun gear 11 with the external toothing 110 is in engagement with external teeth 120 of a plurality of planet gears 12, which are force-transmittingly coupled to the sun gear 11. The planet gears 12 are arranged within a ring gear 14, on the inner circumference of which an internal toothing 140 is formed in order to mesh with the external teeth 120 of the planet gears 12. The planet gears 12 are arranged rotatably on a planet carrier 13 which, via a bearing, e.g. a needle bearing or a plain bearing, is mounted on the drive shaft 2 coaxially with respect to the axis of rotation of the drive shaft 2. In the illustration in FIG. 1, the drive shaft 2 is driven at the left-hand end, wherein the drive shaft 2 is mounted in a housing in a ball bearing 60. The ring gear 14 is likewise mounted in the housing. The drive shaft 2 protrudes into the ring gear 14; the planet carrier 13, through which the drive shaft 2 also protrudes, is arranged axially in front of the ring gear 14. Axially in front of the planet carrier 13, an output section 15 is formed which, in the exemplary embodiment shown, has a larger outer diameter than the external toothing 110 of the sun gear 11. As an alternative to a design of the planetary gear set 10 as a gearwheel mechanism, it is possible to design it also as a friction gear. Instead of the external teeth 110, 120 on the sun gear 11 and the planet gears 12 and the internal toothing 140 in the ring gear 14, the planet gears 12 then run on untoothed surfaces.

The planet gears 12 are mounted on the planet carrier 13 rotatably on journals. On the side of the planet carrier 13 directed away from the planet gears 12 and the ring gear 14, an annular space is formed in which a sleeve-shaped output element 20 is mounted. The output element 20 has an external bearing which, in the exemplary embodiment shown, is designed as a needle bearing or roller bearing and is arranged between the outer inner circumference of the annular space. On the outer side, the output element 20 has form-fit elements in order to engage with the component that is to be driven. Gearwheels, gearwheel segments or a toothing can also be arranged or formed on the outer side. Two freewheels 30, 40 are arranged within the sleeve-shaped output element 20, wherein the first freewheel 30 is arranged between the output section 15 of the drive shaft 2 and the output element 20, while the second freewheel 40 is arranged between the outer side 1333 of a sleeve section 133 of the annular space of the planet carrier 13. The mode of operation of the freewheels 30, 40 will be explained later. The freewheels 30, 40 have clamping bodies 32, 42 which bear on, and can thus be brought into engagement with, the inner side 24 of the output element 20 and the outer side of the output section 15 and of the sleeve section 133. The clamping bodies 32, 42 are arranged substantially concentrically about the axis of rotation of the drive shaft 2 and are arranged axially one behind the another, wherein the clamping bodies 32 of the first freewheel 30 are arranged in a plane in front of the plane of the clamping bodies 42 of the second freewheel 40. The clamping bodies 32, 42 are traversed by shifting pins 52, which are attached to a carrier 50, the latter being mounted on the drive shaft 2 via a bearing 60. In the exemplary embodiment shown, the shifting pins 52 are formed as separate elements, which are fastened in the carrier 50; alternatively, a one-piece configuration of shifting pins 52 and carrier 50 is present. In the exemplary embodiment shown, the carrier 50 is coupled to an actuator 80, which can be a force accumulator or a motor drive, via which the position of the carrier 50 relative to the output element 20 and to the clamping bodies 32, 42 can be adjusted. The actuator 80 can also be a return spring which ensures that a clamping body 32, 42 is always in force-transmitting engagement, or which serves to ensure that the clamping bodies 32, 42 are moved to/held in an engagement position and/or a release position.

The freewheels 30, 40 are activated in the direction of rotation counter to the drive shaft 2 so that, upon rotation in a first direction of rotation, a first freewheel 30 is force-transmittingly activated and the output element 20 is brought into clamping and force-transmitting engagement with the drive shaft 2. This makes it possible to transfer the speed of the drive shaft 2 directly to the output element 20. In the opposite direction of rotation, the second freewheel 40 can be activated in such a way that, after the transmission via the planet gears 12, the planet carrier 13 is force-transmittingly coupled to the output element 20, so that a rotation of the output element 20 with a greater torque and a lower speed is achieved on account of the transmission by the planetary gear set.

FIG. 2 shows a perspective view of the transmission from FIG. 1, without the carrier 50. The drive shaft 2 has a square shaft end in order to be able to couple a drive thereto. The bearings 60 for the bearing in the housing and the bearing of the carrier 50 can be seen on the outer side of the drive shaft 2. Axially behind the bearings 60, the output section 15 of the drive shaft 2 can be seen; clamping bodies 32 of the first freewheel 30 slide along on the outer side of said output section. Shifting pins 52 pass through the clamping bodies 32 and are in turn fastened in the driver, which is not shown. All the clamping bodies 32 of the first freewheel 30 are traversed by a respective shifting pin 52 and, by rotation in one or the other direction, can be adjusted in the circumferential direction relative to the output element 20 and brought into engagement or out of engagement with the drive shaft 2 and the output element 20. The form-fitting elements for defining further output elements or for transmitting force to other components are arranged on the outer side of the output element 20.

Within the output element 20, receiving pockets 22 are formed which are sawtooth-shaped and increase in size radially in the circumferential direction. If the shifting pins 52 are turned to the right, clockwise, the clamping bodies 32 move to the right and in the direction of a region of the receiving pockets 22 that has a greater distance to the output section 15 of the drive shaft 2. If the carrier 50 is turned to the left with the shifting pins 52, the clamping bodies 32 are pressed against the drive shaft 2 onto the output section 15 by the receiving pockets 22 that taper in the radial direction. If the drive shaft 2 is turned counterclockwise, the clamping bodies 32 are clamped between the output section 15 and the receiving pockets 22 of the output element 20 and move the output element 20 counterclockwise. In a reverse direction of rotation, clockwise, the clamping elements 32 are moved in the direction of that end of the receiving pockets 22 of radially increased size, wherein, on account of the increased distance, no clamping effect arises in this direction and the clamping bodies 32 are brought out of engagement. The clamping bodies 32 can also be maintained out of engagement via the shifting pins 52.

The second freewheel 40 is located axially behind the first freewheel 30 and within the planet carrier 13. The mode of operation of the second freewheel 40 corresponds to that of the first freewheel 30, only with a reverse direction of rotation.

FIG. 3 shows the transmission without the housing with the components of the planet carrier in the assembled state. The carrier 50 is arranged on the bearing, and the shifting pins protrude through the clamping bodies both of the first freewheel and the second freewheel.

FIG. 4 shows the transmission without the ring gear. It can be seen that the planet gears 12 are rotatably mounted on journals on the planet carrier 13 and transfer the torque from the sun gear 11 to the ring gear (not shown) by the external teeth or by non-positive transmission of force. By rolling on the inner circumference of the ring gear, the planet carrier 13 is rotated at a speed lower than the drive shaft 2.

A front view of an embodiment similar to FIG. 2 is shown in FIG. 5, in which not all of the clamping bodies 32 of the first freewheel 30 are shown, so as to allow the clamping bodies 42 of the second freewheel 40 to be seen. The clamping bodies 32, 42 of the two freewheels 30, 40 are arranged on the shifting pins 52, which protrude perpendicularly into the drawing plane. All of the shifting pins 52 are connected to one another via the carrier 50 (not shown) and synchronize the adjustment movement of all the clamping bodies 32, 42. Oblong holes 320, 420 are arranged in all of the clamping bodies 32, 42 and allow a radially outward movement of the clamping bodies 32, 42 and a tilting movement about the shifting pins 52. In the exemplary embodiment in FIG. 5, the receiving pockets 22 are provided with a contour which increases radially in size in a direction of rotation, wherein the clamping bodies 32, 42 have an outer contour which corresponds to the contour of the receiving pockets 22 but is increased radially in size. The receiving pockets 22 increase in size in the radial direction, depending on the respective angular position. In the axial direction, the receiving pockets 22 are the same for all section surfaces in the axial extent. The receiving pockets 22 for the first freewheel 30 can also be designed like a thread and taper in the axial direction so that, upon counterclockwise rotation of the shifting pins 52, the clamping bodies 32 are rotated to the left relative to the output element 20, whereby the clamping bodies 32 are also moved perpendicularly to the drawing plane. In all embodiments, the clamping bodies are pressed in the direction of the outer side of the output section 15 by virtue of the reductions in diameter of the sawtooth-like receiving pockets 22.

Radial mobility is afforded by the oblong holes 320 in the clamping bodies 32. As soon as the surface of the clamping body 32 facing the output section 15 comes into contact with the output section 15 of the drive shaft 2, the clamping body 32 is rotated and pivoted about the longitudinal extent of the shifting pin 52, as is indicated by the arrow 321. This causes the clamping body 32 to clamp with the output element 20 and the drive shaft 2 and, in this embodiment, the output element 20 also moves clockwise when the drive shaft moves in the clockwise direction. In addition, the output element 20 rotates counterclockwise when the drive shaft moves counterclockwise, provided that the clamping bodies 32 bear with their inwardly facing surfaces against the output section 15 and that the shifting pins 52 allow sufficient radial displacement inward.

The clamping bodies 42 of the second freewheel 40 are formed correspondingly and oriented in an opposite direction and, in the illustrated position of the shifting pins 52, are disengaged from the outer side 1333 of the sleeve section 133 of the planet carrier 13 and thus cannot transfer forces or torques. When the direction of rotation is reversed and the position of the shifting pins 52 is correspondingly adjusted, the clamping bodies 42 of the second freewheel 40 are brought into contact with the planet carrier 13 and, with a lower speed and a higher torque, transfer the drive power of the drive to the output element 20.

A variant of the transmission is shown in FIG. 6 which, in terms of its basic structure, corresponds to the transmission of FIG. 5. FIG. 6 shows the two contradirectional, sawtooth-shaped outer surfaces of the receiving pockets 22, which outer surfaces increase in size radially outward in different directions. In the exemplary embodiment according to FIG. 6, the clamping bodies 32, 42 are formed as rollers; the receiving pockets 22 have no spirally tapering contact surfaces but are oriented substantially parallel to the longitudinal extent of the shifting pins 52. When the output section 15 of the drive shaft 2 is rotated, the clamping bodies 32 of the first freewheel are pressed against the tapering surfaces of the receiving pockets 22 and move the output element 20 counterclockwise. Upon clockwise rotation of the output section 15, the clamping bodies 32 of the first freewheel 30 are displaced, on account of the oblong hole, in the direction of that end of the receiving pockets 22 increasing in size, and they optionally roll on the shifting pins 52 and do not transmit any forces to the output element 20. By contrast, the clamping bodies 42 of the second freewheel 40 engage with the outer side 1333 of the sleeve section of the planet carrier 13 and clamp the output element 20, so that an adjustment movement of the output element 20 results in a clockwise direction. Changing the direction of rotation activates or deactivates the other freewheel. The two freewheels 30, 40 are coupled via the shifting pins 52 of the carrier 50. If no active adjustment of the shifting pins 52 is to be made via a motor or another controlled drive, it should be ensured that a starting position of a freewheel is always present as standard, so that one freewheel is always coupled and one freewheel always disengaged. For this purpose, the shifting pins 52 and the carrier 50 may be pretensioned with a spring which, for example, always loads the first freewheel in a direction in which it is coupled and keeps the second freewheel always disconnected, or vice versa. The pretensioning force is overcome by the clamping bodies if a corresponding direction of rotation of the drive shaft 2 is present. In such a standard setting, complete decoupling of drive and output is not possible. Complete and permanent decoupling is achieved via the actuator 80 in the active adjustment by virtue of the fact that a central position of the shifting pins 52 is adopted, so that no forces can be transferred from the drive shaft 2 to the output element 20. Such a central position is preferably only possible for a small adjustment path of the carrier 50, in order to enable a quick change-over and activation of the transmission. Active decoupling, or decoupling of the transmission, allows the drive to be completely separated from the orthopedic device. It is thereby possible to provide a purely passive mode of the orthopedic device, for example if both an active drive and a passive influence on the movement behavior of the orthopedic component are provided, for example via a hydraulic damper. Decoupling will also reduce the noise generation, since no motor noises or transmission noises occur additionally to the sounds of the orthopedic device with a purely passive resistance device.

### LIST OF REFERENCE SIGNS

2—Drive shaft
10—Planetary gear set
11—Sun gear
12—Planet gears

113—Planet carrier
14—Ring gear
15—Output section
20—Output element
22—Receiving pockets
24—Inner side
30—Freewheel
32—Clamping body
40—Freewheel
42—Clamping body
50—Carrier
52—Shifting pins
60—Bearing
80—Actuator
110—External toothing
120—External toothing
133—Sleeve section
140—Internal toothing
320—Oblong hole
420—Oblong hole
1333—Outer side

1. A transmission for orthopedic devices, with a planetary gear set (10), which has a central sun gear (11), with a plurality of planet gears (12) which are rotatingly mounted on a planet carrier (13) and are force-transmittingly coupled to the sun gear (11), and with a ring gear (14), which has an inner circumference, in which the planet gears (12) circulate, the sun gear (11) is coupled to a drive shaft (2), and the planet carrier (13) is coupled to an output element (20), characterized in that the output element (20) is coupled to two contradirectionally oriented, shiftable freewheels (30, 40), wherein, in a first direction of rotation of the drive shaft (2), a first freewheel (30) can be brought directly into force-transmitting engagement with the drive shaft (2), and, in a second direction of rotation of the drive shaft (2), counter to the first direction of rotation, a second freewheel (40) can be brought into force-transmitting engagement with the planet carrier (13), in order to drive the output element (20).

2. The transmission as claimed in claim 1, characterized in that the freewheels (30, 40) have clamping bodies (32, 42) which, in an engagement position, come into force-transmitting engagement with the output element (20) and the drive shaft (2) or the planet carrier (13), and the clamping bodies (32, 42) are assigned shifting pins (52) which move the clamping bodies (32, 42) from the engagement position to a release position, in which the clamping bodies (32, 42) are disengaged, and back.

3. The transmission as claimed in claim 2, characterized in that the shifting pins (52) are arranged on a rotatably mounted carrier (50).

4. The transmission as claimed in claim 2 or 3, characterized in that the freewheels (30, 40) are arranged coaxially and axially one behind the other, and on at least one shifting pin (52) there are arranged a clamping body (32) for the first freewheel (30) and a clamping body (42) for the second freewheel (40).

5. The transmission as claimed in claim 3 or 4, characterized in that the carrier (50) is coupled to an actuator (80).

6. The transmission as claimed in any one of claims 3 to 5, characterized in that the clamping bodies (32, 42) and the carrier (50) are designed to hold all the clamping bodies (32, 42) in the release position.

7. The transmission as claimed in any one of the preceding claims, characterized in that the clamping bodies (32) of the first freewheel (30) are arranged between an output section (15), arranged or formed on the drive shaft (2), and an inner side (24) of the output element (20).

8. The transmission as claimed in any one of the preceding claims, characterized in that the output element (20) is rotatably mounted in an inner circumference of the planet carrier (13), and the clamping bodies (42) of the second freewheel (40) are arranged between an inner side of the output element (20) and an outer side (1333) of a sleeve section (133) of the planet carrier (13).

9. The transmission as claimed in any one of the preceding claims, characterized in that the planetary gear set (10) is designed as a friction gear or gearwheel mechanism with external teeth (110, 120) for the sun gear (11) and the planet gears (12) and with an internal toothing (140) for the ring gear (14).

10. The transmission as claimed in any one of the preceding claims, characterized in that receiving pockets (22) increasing radially in size in the circumferential direction and provided for the clamping elements (32, 42) are formed in the output element (20), and the increase in size of the receiving pockets (22) for the clamping elements (32) of the first freewheel (30) runs counter to that of the

11. The transmission as claimed in claim 2, characterized in that the clamping bodies (32, 42) have oblong holes (320, 420) for receiving the shifting pins (52).
